SEQUENCE LISTING (3 sequences)

SEQ ID NO: 1 - DNA, 6916 bp, Neurospora crassa
ccaccaccaccaccaccaccacaacaaccaactcaacaacgaactcaacaaccaaccccaaacccaacctcgacctcaacctcaacctcaacccttgcgacctcgagatacacaaacacatcctcgacaaatgacgcccgacccgctacacgccaacagattgcccccgaggtaggagcatcgacgcatcaggattcagttggacttggagaaggaggaggaggaggaatggcgaagctctcagtcaagaacaacctgccacggccgcacttggtctccttgtcgtcgtcaacgacaggctctgggtctggttctgcgtctaggtcagcttctgctaagcacggaagtgccggttccagtacctttgatcatgaacaacatcaacaacatcaacaacaacaacaacaaaagcgccagcggtcgcaatcagaagcacgacaacagcagcagcaacagcaacagcaacagcaacagcaacaacaacaacaacaagcacagcaccatgcacattctacatatgcacaaagaccccaacccaccccccaacaacgaccaccccaaaacctactgacacctgcttcaaccactggtgccagcgtcggcccgctccaacgcgcatactcggtttcattagctgcgagacagtccccctcgacaaacttggtccgtccaaagaccgactcgccagctccccacactttacacctcaagaacaagaagaacctccgtcaccccgcccccacgcccgacagtccgatcgtagacgacgatattttctccgacgccgtcgatcttaccgaagaactcgatcatgaccatgatctcaacggcaaagacaaagacaacaccgacaacgacaacacagtcgcttccagttcgctaatagggttcggcgatgacaagttactgtggcgagaggactttgctgagcgtgcagagcccgaacatgaaagaggtgggagcaggcctcgccaggtcaagaaacggaagatatcgaatgactacattatgaaggatgaggatgtctcgctttttgatgatgatggcgaggaggacgagtttatggatatcaatgagctagttcagggggatcgggaaagtactccgaagccaaaggctacatcgaggtctgtctcgacgaggctgccgcctacagtatcgctgcaacggggtcggtctcctaagaggaaggaggcttcagttgaaaagcgcacaacggaaaaccagcaacaggctgacagagaagacgaaccgtcgtttatgtcaagtccagatgtcgacaactcccgcaagcgaaagtcttctggatcgcccacaggtttaacgacgccaagaccccagcagaagcaaacggaagaggtcccaggtacgaccaccgccaagaagccacggcgcagtgaagtgatggactcggaggacgaggcattcactcctctttctgctgggtcgctgcctgggagtgcggagttcttcagaagcggtgggaccaccacacgggaattgggtttggacgaagacacggttatggacacgcctagtaggccaccggtcgagtccactttgccaactctcgagtctgtggaaagtcgaccaccccccctgccgcccatggatctaccatcacagcgaaaaccgctagagccgttgaacactccgcgcaaccagctgcttgagtcggtcgaaaggccaacacagcagccgtcggtggggccgagttttgcacagagtagcacactcgccgaaagctccctgccgccgtcaatgccgccgccaagtgaagaccccctcaacaccagggagaacagcaaccttgaggagttcgactacaagctttacaaacccctgctagatcttttcgtcaacgcacccgcaatcttggaaagagaactgagcgccgttaatgacgagcttcaggagaacatgatcaagctgcgggactgtctgcgcctgcccagggaagaaagagacagggcacgcgaagaggtgaagaaggaaaaggaaatgctcaagcgacgggacattgcgctcagagccctccaggacgaacacaagttgtacgtcaagaaacgcaaagagcataatttgatcaacgaggaaatcgttcgcgcttatgctgaagaagacgatgagtacgaggatcagttaatggcgcagctggacaagttggatgatgaggttgaggctatcgtaaagagtctgacaaggcttattgtggcggcggggatcacggagaagagctttgacctaaagaaggaggaggaagaggaggaggagaagccgatcatcatagcgactccgacaccttcgacgaggaccgaggccccggttctgccgacgaccgagtatcataattcccagcaggtcatattgcagactcaacatcctgctgcgcagcaggtttctcaccgggtgccaccacctccgacaccgagttttcaaacggcgcgccagactccggtgtcatatcagagcagaccgaccaacaactcctttcctgatatctcggcggaagaagccatgatgttcgataaagaagaccccttcatggaacaacagcacgccccggcctctgctcccttccaggccacccttccccagcgcaacagccctttcaaaaccgccccgttcaagccagtccacggccacgattactttgacgatgaagacgacgatgccgacctcctggcagcagtagacagcgccgagacgtatacttctacggccgccaccaccaccaccaacaacaacaatcacttacgatcacaatcggtgatgtcaacatccacggcgaccacgatcaaaccgaggaaacgcaacgaaaatgccaatgccaagaagcccaagtccgtacatgcaaagttatcgatgccgcccgaaaagatgaagtatgcgtggtcgaatgatgtgaggaaggctctcaaggataggtttcggatgtcggggttcagacagaatcagttggaggctattaatgctactttgggtggtaaggtgagttctctgtcctttacctatctgggagagaccaagaaggagagagagagagagaggaggggaagacgaaaatggactttgctgactctagaaaggatgcctttgtgttgatgccgactggtggtggaaagtctctgtgctatcagttgccggctgtagtcaggagcggcaagacgcgtggtatcacagtcgtcatctcccctctgctaagtctgatgctggatcaagtcaaccatttggcaaacctgatgatccaagcttacgctttcaacggagacatgaactcagaaatgcgccgaatggtgtttcagaagcttgatgctgagcatcctgagcatgagctccaactgctctatgtcaccccggagatggtgagcaagaaccagacattcgtcaacaagatgatggacctctaccggaggaaaaagctggctagaattgttatcgacgaggctcactgcgtcagtcaatggggccatgacttccgacccgattacaaagctataggagagtttcgtaagaggtttcccggagttccggtcatggctttgacagcgacggcaacacagaacgtcatcttggatgtcaagcataacctggcaatggaggactgccagactttctcccagagctttaatcggccgaacctctactatgaggtcaggatgaaggagcagaatctgattgcccgcatcgcagagttgatcaaggagaagtatgacggccagacgggtatcatctacacattatcaagaaagagtgccgagaacatcgccaaaaatctccaggaaaaacaccgcatcaaagcaaagcactaccatgcctccatcaccaccgacgaaaagatcagcgttcaacatgaatggcaaaccggccgagtcaaagtcgtggtagccaccattgccttcggcatgggcatcgacaagcctgacgtccgctttgttatccaccagcacatccccaagtcgctcgaaggttactatcaagaaaccggccgcgccggacgtgacggcaagccatcggactgctacttgtactttgcctatggcgacattcaatccctacgtcgtatgatcgccgacggcgaaggtgactacgcgcaaaaggagcgtcagctacaaatgctcaaccgtgtggtcagctattgcgagtcgcagcacacgtgccggcgcgaagaagtgctccgctactttggcgaggagtttgactaccggaagtgtagagacggatgcgataactgccggaacggacgcatctcgaagtcgacggagatgagggattttacggaaatcgccttcgccgcgatcgaggtggtgaagagccagcagcccatcacgctgggcaagctgtgcgacatcctgatgggcaagagaaagaacgagcacggtggcgtgtgtcactttggtatcgccaaggggagcacgcagagggagctgcagaggatcgtgctgcagctgaatttccacaaggcgctgggcgaggacaatatcatgaatggggcggggatgcctattacctactatattgtgagtgctgtcccggttggtcttgcatatctggctttgttgctttgctaacacagcagctcgtacagaccggccctgaagctggtgcttacctctacaatggcaagcggttgatgctgccagttccctcaaacaagtccgtcgaacccccgtctcggtctaagcagcggagccgtcgagtcgatgaggatatggatgagcaagaactttccaccctgcaacgaccgccaacatcaacaaatgtctcttcacccgttcgagccaccaagaaacgaagttccaaaaaggctttaccgaccctcatcgccgactacgaagagcccagctccgacggtcctcacggtcctctccacgccaacggctatgagcgcgataactttgtcgtatccgataatgttgaacccgaagaggaagaagatgccttcgaacctgtccgcccctcgcggcgcggcccatcttctcgcgctacccgccctcaacaccgccagaccaccctttatgacaccctctcccacacccaacaatcccaaaccgtctcccaacacctcgccactttgggtccgcccatcgacgcccgcaccatgcataacccccgctacgcccagcttgacgaggtccaccaggatattgtcgatgcctttgttgaagaagtcaaggtcttcgaggaggactttcgcaacaggaaccacatgcgcaaacccatctttaccgagacgcagtaccgtgagatggcaatccggtggacgcggtcgttagacgcgatgcgcgcgatcccggatatcaaccaggataaagtagatcggtatggtgccaaattcatcccacttgtggagcggttctgggggaattatcaggagatgatggggggagggtatgataatcctgctgtggctggcgatgaggatgatgatgagggccccaggaggacaggaaatggaaaaggggggaataagaagggaggaggaggaggaggaggaaatgaagtagtggatttgattagtagtgatgaggatgaacctccggctcgtgcaccatcgcggaatgcggggcgaggaaaggcacagtcgacacgtgggggacaaatccaagataaaggccgagcagtcaaccgccgcggagaacccatcgccgaagaagacgaagaagactacgggctaagcgaccccgatatcgacgccatcgatccagacgccatcaccgcctccgacaactccgacgaagaagatgatgatgatgatgacgaagacctcgaatcctcccgctacttctccggctcaacaggcccgcccgtctccaaagccgtgcaggatgctcgactccgtgaacaactttccatgtacgcctccggcggcagctcttcgaaaggtagctacggctcagggcgcgcatcaggaggatcttcgtcgagagcgtcgggatcaggatggagaggtggaggagcaggtgggaagaaatactacaggaagaagagggctggttcttcggctgctggtggtggtggtgcaggaggagggggagttacaaaacggaaggcgagtgggagtggcgcgaagacggcgaggaagaggggtgcatctactgcgccgaagacaacgacgagagggggaggatctggagctgggtctagaggaggcggtgctggtggtgctggtggtgctggtgctggtgctggtgctggaggagggaaaaggggtggtggaggtggaggaggaatgggagggataagtgttatgcctcattagctattttatagcatatcgcatttatacagtgtcttatggaagggaggaggagaagaagaaggataagctggcataagcttgaaccggccaggccaaaatggccagagagctcaccgggcaatcgagcttgaaatgagcttgacatattaggtattcccgagaatatagcgggattacaaggcacttactttaccaagtcgaaagggacgagccaaatctatggtactcgccagttgcgcaacgttgagttttatcattcgtggagttttcatcgtggagtttttattatcaactattcgttgtatagttttcgttgtagatgttagttccggacgatcaaaaggggaagtgtggaacagagaagtcgaaaggacaagccaaaatgacatggcagtgtccagtcagataccctccagacaaaaccagacaccaataacaaacccttcaaccataacaccagcaaagccaatccttaggtacctacctagggtagggtaggtccaggaatgtcttccccaaaggtacctctacttattcatgttacgctccatcagtcccatcgcttagcatcgctgcccggttacctatctctacctctacctctacctctacctctacctctacctctacctctatctctacctctacctctac

SEQ ID NO: 2 - DNA, 5868 bp, Neurospora crassa
  CDS (1)..(5868)
atggcgaagctctcagtcaagaacaacctgccacggccgcacttggtctccttgtcgtcgtcaacgacaggctctgggtctggttctgcgtctaggtcagcttctgctaagcacggaagtgccggttccagtacctttgatcatgaacaacatcaacaacatcaacaacaacaacaacaaaagcgccagcggtcgcaatcagaagcacgacaacagcagcagcaacagcaacagcaacagcaacagcaacaacaacaacaacaagcacagcaccatgcacattctacatatgcacaaagaccccaacccaccccccaacaacgaccaccccaaaacctactgacacctgcttcaaccactggtgccagcgtcggcccgctccaacgcgcatactcggtttcattagctgcgagacagtccccctcgacaaacttggtccgtccaaagaccgactcgccagctccccacactttacacctcaagaacaagaagaacctccgtcaccccgcccccacgcccgacagtccgatcgtagacgacgatattttctccgacgccgtcgatcttaccgaagaactcgatcatgaccatgatctcaacggcaaagacaaagacaacaccgacaacgacaacacagtcgcttccagttcgctaatagggttcggcgatgacaagttactgtggcgagaggactttgctgagcgtgcagagcccgaacatgaaagaggtgggagcaggcctcgccaggtcaagaaacggaagatatcgaatgactacattatgaaggatgaggatgtctcgctttttgatgatgatggcgaggaggacgagtttatggatatcaatgagctagttcagggggatcgggaaagtactccgaagccaaaggctacatcgaggtctgtctcgacgaggctgccgcctacagtatcgctgcaacggggtcggtctcctaagaggaaggaggcttcagttgaaaagcgcacaacggaaaaccagcaacaggctgacagagaagacgaaccgtcgtttatgtcaagtccagatgtcgacaactcccgcaagcgaaagtcttctggatcgcccacaggtttaacgacgccaagaccccagcagaagcaaacggaagaggtcccaggtacgaccaccgccaagaagccacggcgcagtgaagtgatggactcggaggacgaggcattcactcctctttctgctgggtcgctgcctgggagtgcggagttcttcagaagcggtgggaccaccacacgggaattgggtttggacgaagacacggttatggacacgcctagtaggccaccggtcgagtccactttgccaactctcgagtctgtggaaagtcgaccaccccccctgccgcccatggatctaccatcacagcgaaaaccgctagagccgttgaacactccgcgcaaccagctgcttgagtcggtcgaaaggccaacacagcagccgtcggtggggccgagttttgcacagagtagcacactcgccgaaagctccctgccgccgtcaatgccgccgccaagtgaagaccccctcaacaccagggagaacagcaaccttgaggagttcgactacaagctttacaaacccctgctagatcttttcgtcaacgcacccgcaatcttggaaagagaactgagcgccgttaatgacgagcttcaggagaacatgatcaagctgcgggactgtctgcgcctgcccagggaagaaagagacagggcacgcgaagaggtgaagaaggaaaaggaaatgctcaagcgacgggacattgcgctcagagccctccaggacgaacacaagttgtacgtcaagaaacgcaaagagcataatttgatcaacgaggaaatcgttcgcgcttatgctgaagaagacgatgagtacgaggatcagttaatggcgcagctggacaagttggatgatgaggttgaggctatcgtaaagagtctgacaaggcttattgtggcggcggggatcacggagaagagctttgacctaaagaaggaggaggaagaggaggaggagaagccgatcatcatagcgactccgacaccttcgacgaggaccgaggccccggttctgccgacgaccgagtatcataattcccagcaggtcatattgcagactcaacatcctgctgcgcagcaggtttctcaccgggtgccaccacctccgacaccgagttttcaaacggcgcgccagactccggtgtcatatcagagcagaccgaccaacaactcctttcctgatatctcggcggaagaagccatgatgttcgataaagaagaccccttcatggaacaacagcacgccccggcctctgctcccttccaggccacccttccccagcgcaacagccctttcaaaaccgccccgttcaagccagtccacggccacgattactttgacgatgaagacgacgatgccgacctcctggcagcagtagacagcgccgagacgtatacttctacggccgccaccaccaccaccaacaacaacaatcacttacgatcacaatcggtgatgtcaacatccacggcgaccacgatcaaaccgaggaaacgcaacgaaaatgccaatgccaagaagcccaagtccgtacatgcaaagttatcgatgccgcccgaaaagatgaagtatgcgtggtcgaatgatgtgaggaaggctctcaaggataggtttcggatgtcggggttcagacagaatcagttggaggctattaatgctactttgggtggtaaggatgcctttgtgttgatgccgactggtggtggaaagtctctgtgctatcagttgccggctgtagtcaggagcggcaagacgcgtggtatcacagtcgtcatctcccctctgctaagtctgatgctggatcaagtcaaccatttggcaaacctgatgatccaagcttacgctttcaacggagacatgaactcagaaatgcgccgaatggtgtttcagaagcttgatgctgagcatcctgagcatgagctccaactgctctatgtcaccccggagatggtgagcaagaaccagacattcgtcaacaagatgatggacctctaccggaggaaaaagctggctagaattgttatcgacgaggctcactgcgtcagtcaatggggccatgacttccgacccgattacaaagctataggagagtttcgtaagaggtttcccggagttccggtcatggctttgacagcgacggcaacacagaacgtcatcttggatgtcaagcataacctggcaatggaggactgccagactttctcccagagctttaatcggccgaacctctactatgaggtcaggatgaaggagcagaatctgattgcccgcatcgcagagttgatcaaggagaagtatgacggccagacgggtatcatctacacattatcaagaaagagtgccgagaacatcgccaaaaatctccaggaaaaacaccgcatcaaagcaaagcactaccatgcctccatcaccaccgacgaaaagatcagcgttcaacatgaatggcaaaccggccgagtcaaagtcgtggtagccaccattgccttcggcatgggcatcgacaagcctgacgtccgctttgttatccaccagcacatccccaagtcgctcgaaggttactatcaagaaaccggccgcgccggacgtgacggcaagccatcggactgctacttgtactttgcctatggcgacattcaatccctacgtcgtatgatcgccgacggcgaaggtgactacgcgcaaaaggagcgtcagctacaaatgctcaaccgtgtggtcagctattgcgagtcgcagcacacgtgccggcgcgaagaagtgctccgctactttggcgaggagtttgactaccggaagtgtagagacggatgcgataactgccggaacggacgcatctcgaagtcgacggagatgagggattttacggaaatcgccttcgccgcgatcgaggtggtgaagagccagcagcccatcacgctgggcaagctgtgcgacatcctgatgggcaagagaaagaacgagcacggtggcgtgtgtcactttggtatcgccaaggggagcacgcagagggagctgcagaggatcgtgctgcagctgaatttccacaaggcgctgggcgaggacaatatcatgaatggggcggggatgcctattacctactatattaccggccctgaagctggtgcttacctctacaatggcaagcggttgatgctgccagttccctcaaacaagtccgtcgaacccccgtctcggtctaagcagcggagccgtcgagtcgatgaggatatggatgagcaagaactttccaccctgcaacgaccgccaacatcaacaaatgtctcttcacccgttcgagccaccaagaaacgaagttccaaaaaggctttaccgaccctcatcgccgactacgaagagcccagctccgacggtcctcacggtcctctccacgccaacggctatgagcgcgataactttgtcgtatccgataatgttgaacccgaagaggaagaagatgccttcgaacctgtccgcccctcgcggcgcggcccatcttctcgcgctacccgccctcaacaccgccagaccaccctttatgacaccctctcccacacccaacaatcccaaaccgtctcccaacacctcgccactttgggtccgcccatcgacgcccgcaccatgcataacccccgctacgcccagcttgacgaggtccaccaggatattgtcgatgcctttgttgaagaagtcaaggtcttcgaggaggactttcgcaacaggaaccacatgcgcaaacccatctttaccgagacgcagtaccgtgagatggcaatccggtggacgcggtcgttagacgcgatgcgcgcgatcccggatatcaaccaggataaagtagatcggtatggtgccaaattcatcccacttgtggagcggttctgggggaattatcaggagatgatggggggagggtatgataatcctgctgtggctggcgatgaggatgatgatgagggccccaggaggacaggaaatggaaaaggggggaataagaagggaggaggaggaggaggaggaaatgaagtagtggatttgattagtagtgatgaggatgaacctccggctcgtgcaccatcgcggaatgcggggcgaggaaaggcacagtcgacacgtgggggacaaatccaagataaaggccgagcagtcaaccgccgcggagaacccatcgccgaagaagacgaagaagactacgggctaagcgaccccgatatcgacgccatcgatccagacgccatcaccgcctccgacaactccgacgaagaagatgatgatgatgatgacgaagacctcgaatcctcccgctacttctccggctcaacaggcccgcccgtctccaaagccgtgcaggatgctcgactccgtgaacaactttccatgtacgcctccggcggcagctcttcgaaaggtagctacggctcagggcgcgcatcaggaggatcttcgtcgagagcgtcgggatcaggatggagaggtggaggagcaggtgggaagaaatactacaggaagaagagggctggttcttcggctgctggtggtggtggtgcaggaggagggggagttacaaaacggaaggcgagtgggagtggcgcgaagacggcgaggaagaggggtgcatctactgcgccgaagacaacgacgagagggggaggatctggagctgggtctagaggaggcggtgctggtggtgctggtggtgctggtgctggtgctggtgctggaggagggaaaaggggtggtggaggtggaggaggaatgggagggataagtgttatgcctcattag

SEQ ID NO: 3 - protein, 1955 aa, Neurospora crassa
MAKLSVKNNLPRPHLVSLSSSTTGSGSGSASRSASAKHGSAGSSTFDHEQHQQHQQQQQQKRQRSQSEARQQQQQQQQQQQQQQQQQQAQHHAHSTYAQRPQPTPQQRPPQNLLTPASTTGASVGPLQRAYSVSLAARQSPSTNLVRPKTDSPAPHTLHLKNKKNLRHPAPTPDSPIVDDDIFSDAVDLTEELDHDHDLNGKDKDNTDNDNTVASSSLIGFGDDKLLWREDFAERAEPEHERGGSRPRQVKKRKISNDYIMKDEDVSLFDDDGEEDEFMDINELVQGDRESTPKPKATSRSVSTRLPPTVSLQRGRSPKRKEASVEKRTTENQQQADREDEPSFMSSPDVDNSRKRKSSGSPTGLTTPRPQQKQTEEVPGTTTAKKPRRSEVMDSEDEAFTPLSAGSLPGSAEFFRSGGTTTRELGLDEDTVMDTPSRPPVESTLPTLESVESRPPPLPPMDLPSQRKPLEPLNTPRNQLLESVERPTQQPSVGPSFAQSSTLAESSLPPSMPPPSEDPLNTRENSNLEEFDYKLYKPLLDLFVNAPAILERELSAVNDELQENMIKLRDCLRLPREERDRAREEVKKEKEMLKRRDIALRALQDEHKLYVKKRKEHNLINEEIVRAYAEEDDEYEDQLMAQLDKLDDEVEAIVKSLTRLIVAAGITEKSFDLKKEEEEEEEKPIIIATPTPSTRTEAPVLPTTEYHNSQQVILQTQHPAAQQVSHRVPPPPTPSFQTARQTPVSYQSRPTNNSFPDISAEEAMMFDKEDPFMEQQHAPASAPFQATLPQRNSPFKTAPFKPVHGHDYFDDEDDDADLLAAVDSAETYTSTAATTTTNNNNHLRSQSVMSTSTATTIKPRKRNENANAKKPKSVHAKLSMPPEKMKYAWSNDVRKALKDRFRMSGFRQNQLEAINATLGGKDAFVLMPTGGGKSLCYQLPAVVRSGKTRGITVVISPLLSLMLDQVNHLANLMIQAYAFNGDMNSEMRRMVFQKLDAEHPEHELQLLYVTPEMVSKNQTFVNKMMDLYRRKKLARIVIDEAHCVSQWGHDFRPDYKAIGEFRKRFPGVPVMALTATATQNVILDVKHNLAMEDCQTFSQSFNRPNLYYEVRMKEQNLIARIAELIKEKYDGQTGIIYTLSRKSAENIAKNLQEKHRIKAKHYHASITTDEKISVQHEWQTGRVKVVVATIAFGMGIDKPDVRFVIHQHIPKSLEGYYQETGRAGRDGKPSDCYLYFAYGDIQSLRRMIADGEGDYAQKERQLQMLNRVVSYCESQHTCRREEVLRYFGEEFDYRKCRDGCDNCRNGRISKSTEMRDFTEIAFAAIEVVKSQQPITLGKLCDILMGKRKNEHGGVCHFGIAKGSTQRELQRIVLQLNFHKALGEDNIMNGAGMPITYYITGPEAGAYLYNGKRLMLPVPSNKSVEPPSRSKQRSRRVDEDMDEQELSTLQRPPTSTNVSSPVRATKKRSSKKALPTLIADYEEPSSDGPHGPLHANGYERDNFVVSDNVEPEEEEDAFEPVRPSRRGPSSRATRPQHRQTTLYDTLSHTQQSQTVSQHLATLGPPIDARTMHNPRYAQLDEVHQDIVDAFVEEVKVFEEDFRNRNHMRKPIFTETQYREMAIRWTRSLDAMRAIPDINQDKVDRYGAKFIPLVERFWGNYQEMMGGGYDNPAVAGDEDDDEGPRRTGNGKGGNKKGGGGGGGNEVVDLISSDEDEPPARAPSRNAGRGKAQSTRGGQIQDKGRAVNRRGEPIAEEDEEDYGLSDPDIDAIDPDAITASDNSDEEDDDDDDEDLESSRYFSGSTGPPVSKAVQDARLREQLSMYASGGSSSKGSYGSGRASGGSSSRASGSGWRGGGAGGKKYYRKKRAGSSAAGGGGAGGGGVTKRKASGSGAKTARKRGASTAPKTTTRGGGSGAGSRGGGAGGAGGAGAGAGAGGGKRGGGGGGGMGGISVMPH

(19) United States
(12) Patent Application Publication
(10) Pub. No.: US 2003/0110520 A1
(43) Pub. Date: Jun. 12, 2003

(54) Isolation and characterization of a N. crassa silencing gene and uses thereof

(76) Inventors: Guiseppe Macino, Roma (IT); Carlo Cogoni, Roma (IT)

Correspondence Address: Samuels, Gauthier & Stevens LLP, Suite 3300, 225 Franklin Street, Boston, MA, 02110, US

(21) Appl. No.: 10/293,504

(22) Filed: Nov. 13, 2002

Related U.S. Application Data
(62) Division of application No. 09/857,091, filed on Aug. 17, 2001.

(30) Foreign Application Priority Data
Nov. 30, 1998 (IT) RM98A000730

(51) Int. Cl. A01K 67/00; C12Q 1/68; C07H 21/04; C12N 9/22; C12N 1/16; A01H 1/00; C12N 15/82

(52) U.S. Cl. 800/8; 800/278; 435/6; 435/69.1; 435/199; 435/254.2; 435/320.1; 536/23.2

(57) ABSTRACT

A nucleotide sequence encoding for a protein characterized in that it has a silencing activity and comprises a recQ helicase domain is disclosed; furthermore expression vectors suitable for expression of said sequence in bacteria, plants, animals and fungi are disclosed; the invention refers also to organisms transformed by such vectors.

The present invention relates to the isolation and characterization of a Neurospora crassa gene encoding for an essential activity in the co-suppression process and to uses and applications thereof in vegetal, animal and fungine fields.

The production of transgenic organisms is of large utility both in basic and applied biological research. The transgenic DNA is usually integrated in the genome and transferred as a Mendelian character. However, in various instances, the transgene introduction induces gene silencing phenomena (Flavell, R. B. 1994), i.e. the repression of the expression of the transgene itself and/or of one or more endogenous homologous genes.

The gene silencing can act at two levels: transcriptional (trans-inactivation) where transgenes contain sequences homologous to the silenced gene promoter (Vaucheret, 1993); and post-transcriptional (co-suppression) which requires homologies between coding regions (Flavell, 1994; Stam et al., 1997; Baulcombe, 1996).

Generally the silencing induced by a transgene requires an almost complete sequence homology (from 70% to 100%) between transgene and silenced gene sequences (Elkind, 1990).

In the Neurospora crassa filamentous fungus, during the vegetative phase, the presence of transgenes induces a post-transcriptional gene silencing phenomenon, named “quelling” (Cogoni et al., 1996).

By using the al-1 gene (albino 1) (Schmidhauser et al., 1990) as silencing visual marker, many features of the phenomenon have been discovered (Cogoni et al., 1996). Particularly the al-1 gene “quelling” in Neurospora is characterized in that: 1) the gene silencing is reversible further to the loss of transgene copies; 2) the reduction of mRNA basal level results from a post-transcriptional effect; 3) transgenes containing at least a region of 132 base pairs which is identical to the region encoding for the target gene are sufficient to induce the “quelling”; 4) the duplication of promoter sequences is ineffective to induce the silencing; 5) the “quelling” exhibits a dominant behavior in eterocarions containing both transgenic and untransformed nuclei, indicating the involvement of a molecule which is acts “in trans” among the nuclei; 6) the expression of an aberrant RNA transcribed by the transgenic locus is strictly correlated to silencing, suggesting that the “quelling” can be induced and/or mediated by a transgenic RNA molecule.

Therefore homologies between Neurospora silencing and plant co-suppression can be pointed out. The gene silencing in Neurospora is reversible, as result of transgenic copies instability during mitotic phase; in plants also the co-suppression reversion is associated with the reduction of transgene copy number, resulting from intra-chromosomal recombination during mitosis or meiosis (Mittelstein Scheid et al., 1994; Stam et al., 1998). Thus both in plants and in Neurospora the transgene presence is required to maintain the silencing. As in Neurospora, a decrease of the mRNA basal level of the silenced gene results from a post-transcriptional mechanism (Dehio and Schell 1994; van Blokand et al., 1994; de Carvalho et al., 1995). Furthermore to induce the “quelling”, transgenes must contain a portion of the silencing target gene coding sequence, being the promoter region ineffective. In plants coding regions with no promoter sequences can induce silencing (van Blokand et al., 1994) and, as in the “quelling”, promoters or functionally active gene products are not required for the co-suppression.

One of the similarities between “quelling” and co-suppression in plants is that both mechanisms are mediated by diffusion factors. In Neurospora eterokaryotic strains, nuclei wherein the albino-1 gene is silenced are able to induce the al-1 gene silencing of the other not transformed nuclei, all sharing the same cytoplasmic environment (Cogoni et al., 1996). In plants the presence of a diffusion factor results from the fact that the co-suppression is effective in inhibiting the replication of Tobacco Etch Virus (TEV), a RNA virus with an exclusively cytoplasmic cycle. The occurrence of highly diffusible factors, which are effective to mediate the co-suppression, has been demonstrated using the grafting technique in tobacco (Palaqui et al., 1997), showing that silenced tobacco plants are able to transfer the silencing to non-silenced plants through grafting.

The fact that “quelling” and co-suppression share all these features suggests that mechanisms involved in post-transcriptional gene silencing in plants and in fungi can be evolved by an ancestral common mechanism.

Recently gene inactivation phenomena resulting from transgene introduction have been disclosed in animals. In Drosophila melanogaster the location of a transgene close to heterochromatic centers results in a variegate expression (Wallrath and Elgin, 1995; Pirrotta, V., 1997). Similar expression profiles have been observed when the reference transgene is within tandem arrayed transposons, indicating that tandem repeats are effective to induce the chromatin condensation. (Dorer and Henikoff, 1994). Again in Drosophila Pal-Bhadra et al. (1997) have observed that the transgene introduction can lead to gene inactivation phenomena, similar to the co-suppression.

Gene silencing phenomena resulting from transegene sequence repeats have been disclosed recently in mammalians.

Garrick et al. (1998) produced mouse transgenic lines wherein 100 transgenic copies are present only in a locus and are directly tandem arrayed. The transgene expression has been disclosed to be inversely proportional to the number of occurring copies, indicating that silencing phenomena dependent on repeat copies are present also in mammalians.

Therefore the identification of Neurospora genes which are involved in the silencing is the first step to modulate the same process in plants, animals and fungi. The silencing modulation is of great relevance when transgenic organisms able to express the desired phenotype are produced.

The authors of the present invention have already isolated Neurospora crassa strains having mutations regarding essential functions for gene silencing mechanism (Cogoni and Macino, 1997); 15 independent isolated mutants define three complementation groups, thus identifying the qde-1, qde-2 and qde-3 genes (qde stands for “quelling”-deficient), whose products are essential to the silencing machinery. qde genes are essential to the Neurospora silencing, as suggested by the fact that silencing of three independent genes (al-1, al-2 and qa-2) is impaired by qde mutations (Cogoni and Macino, 1997).

The authors of the invention have identified and cloned now one out of Neurospora qde genes, thus identifying one of required factors for silencing. By considering the similarity between “quelling” and co-suppression, genes orthologous to the isolated gene are involved in co-suppression and more generally in gene silencing in other organisms, like plants, fungi and animals.

The present invention can be applied with reference to two general scope: 1) silencing potentiation as a tool for inactivating more effectively and durably a desired gene, and 2) silencing suppression to obtain a better expression of the introduced transgenes.

As to the silencing potentiation, the over-expression of one or more genes controlling the phenomenon can lead to higher efficiency and/or stability thereof. Therefore the introduction of qde-3 gene or of homologous genes thereof in microorganisms can constitute a tool to repress more effectively gene functions. Particularly this approach is specially useful in plants wherein the co-suppression is usually used for the “knock-out” of gene functions. In plants again the gene silencing potentiation can be used to obtain lines resistant to pathogen virus, by introducing transgenes encoding for viral sequences, in order to achieve the expression inhibition of the virus itself (Flavell et al., 1994).

Analogous applications are suitable for animals, wherein some indications suggest that silencing can inhibit the suitable expression of introduced transgenes (Garrick et al., 1998).

On the contrary, there are instances wherein it is desirable not to have or to reduce the gene silencing, i.e. where a transgene is to be over-expressed. It is known that the co-suppression is strictly correlated both with the presence of an high copy number of the transgene, and with a transgene high expression. This correlation can hamper the production of transgenic organisms which express a transgene at high levels, because more high is the expression and/or the copy number, more probable is to evoke silencing responses. As above mentioned, analogous mechanisms of gene inactivation, dependent on a high copy number, have been disclosed in animals. In these circumstances plant or animal lines, totally or partially ineffective for silencing, constitute an ideal recipient wherein the desired gene can be over-expressed. The invention can be applied within this scope using different approaches:

A) Identification and Production of Mutant Lines in Genes Homologous to qde-3 Gene, in Plants, Animals and Fungi.

The knowledge of Neurospora qde-3 gene, essential for silencing mechanism, can allow the isolation of mutant lines in other organisms, mutated in genes homologous to qde-3. For example by means of amplifications using degenerated primers, designed from the most conserved regions of qde-3 gene, mutant lines in homologous genes can be identified, by analysis of insertion mutant gene banks, already available for many plant species. Both in fungi and animals such mutants can be obtained, following the identification of the homologous gene, by means of “gene disruption” techniques using homologous recombination.

B) Reduction of qde-3 Gene Expression

Other strategies for the production of silencing-deficient lines comprise the use of Neurospora qde-3 gene or homologous genes thereof. qde-3 or homologous genes can be introduced into suitable expression vectors to express them in an anti-sense orientation in order to inhibit the expression of resident endogenous genes. Alternatively portions of qde-3 or of homologous genes can be over-expressed, in order to obtain a negative dominant effect and thus blocking the function of qde-3 endogenous genes.

The authors of the present invention have cloned and characterised the Neurospora crassa qde-3 gene. The sequence analysis showed that qde-3 gene belongs to a highly conserved gene family, from E. coli to humans, named recQ. Genes belonging to this family encode for DNA helicase, as demonstrated by in vitro assays (Gray et al., 1997). The recQ helicase family is involved in recombinant processes. Mutations of these genes produce iper-recombinant phenotypes as, for example, the S. cerevisiae Sgs-1 gene involved both in meiotic and mitotic recombination.

The authors of the invention for the first time have demonstrated that a gene encoding for a recQ DNA-helicase is involved in gene silencing induced by transgenes. Therefore for the first time it is disclosed that a gene belonging to the recQ family, other than acts during recombination, is also an essential component of the inactivation of repeat sequences.

Therefore it is an object of the invention a nucleotide sequence encoding for a protein characterized in having a silencing activity and comprising a recQ helicase domain, wherein the domain is at least 30% homologous with the amino acid sequence from aa. 897 to aa. 1330 of SEQ ID No.1. More preferably said homology is of at least of 60%. Most preferably the recQ helicase domain comprises the amino acid sequence from aa. 897 to aa. 1330 of SEQ ID No.1. According to a particular embodiment the nucleotide sequence encodes for a protein having the amino acid sequence of SEQ ID No. 1, or functional portions thereof. Even more preferably the nucleotide sequence of the invention is the sequence of SEQ ID No. 1 or its complementary sequence.

A further object of the invention is an expression vector comprising, under the control of a promoter that is expressed in bacteria, the nucleotide sequence of the invention. Those skilled in the art will appreciate that any plasmid suitable for a correct and effective expression of the protein of the invention in bacteria can be used and is within the scope of the invention.

A further object of the invention is an expression vector comprising, under the control of a promoter which is expressed in plants or in specific plant organs, the nucleotide sequence of the invention, both in a sense and anti-sense orientation. Those skilled in the art will appreciate that any plasmid suitable for a correct and effective expression of the protein of the invention in plants or in specific plant organs can be used and is within the scope of the invention.

A further object of the invention is an expression vector comprising, under the control of a promoter which is expressed in fungi or in portions thereof, the nucleotide sequence of the invention, both in a sense and anti-sense orientation. Those skilled in the art will appreciate that any plasmid suitable for a correct and effective expression of the protein of the invention in fungi or in portions thereof can be used and is within the scope of the invention.

A further object of the invention is an expression vector comprising, under the control of a promoter that is expressed in animals, the nucleotide sequence of the invention both in a sense and anti-sense orientation. Those skilled in the art will appreciate that any plasmid suitable for a correct and effective expression of the protein of the invention in animals can be used and is within the scope of the invention.

A further object of the invention is a prokaryotic organism transformed by using the expression vector active in bacteria of the invention.

A further object of the invention is a plant or a specific plant organ transformed by using the expression vector active in plants of the invention.

A further object of the invention is a plant mutated at the nucleotide sequence of the invention and having a reduced or inhibited silencing activity.

A further object of the invention is a fungus transformed with the expression vector of the invention active in fungi.

A further object of the invention is a fungus mutated at the nucleotide sequence of the invention and having a reduced or inhibited silencing activity.

A further object of the invention is a non-human animal transformed with the expression vector of the invention active in animals.

A further object of the invention is a non-human animal mutated at the nucleotide sequence of the invention and having a reduced or inhibited silencing activity.

A further object of the invention refers to a protein characterized in having a silencing activity and in comprising a recQ helicase domain, wherein the domain is at least 30% homologous to the amino acid sequence from aa. 897 to aa. 1330 of SEQ ID No.1. Preferably the recQ helicase domain is at least 40% homologous with the amino acid sequence from aa. 897 to aa. 1330 of SEQ ID No.1. More preferably the recQ helicase domain is at least 60% homologous with the amino acid sequence from aa. 897 to aa. 1330 of SEQ ID No.1. Most preferably the recQ helicase domain comprises the amino acid sequence from aa. 897 to aa. 1330 of SEQ ID No.1. According to a particular embodiment the protein comprises the amino acid sequence of SEQ ID. No.1 or functional portions thereof.

It is within the scope of the invention the use of the nucleotide sequence of the invention to modulate gene silencing in plants, animals and fungi.

It is within the scope of the invention the use of the nucleotide sequence of the invention to potentiate the antiviral-response in a plant.

The present invention now will be disclosed by way of non limiting examples with reference to the following figures:

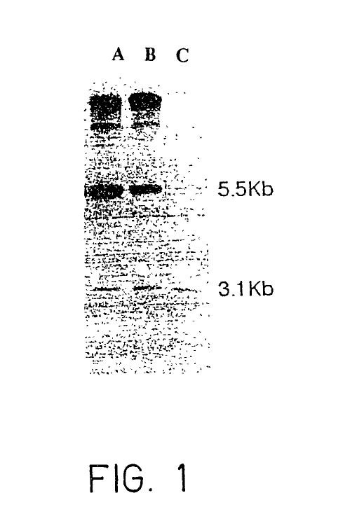

FIG. 1: Southern blot analysis of genomic DNA extracted from (A): untransformed wild type strain, (B): 6xw recipient strain and (C): untransformed wild type strain, SmaI and HindIII digested, blotted and al-1 gene probe hybridized. The 3.1-Kb band corresponds to the endogenous al-1 gene, while the 5.5-Kb band corresponds to tandem arrayed al-1 transgenes. The larger band represents undigested methylated DNA.

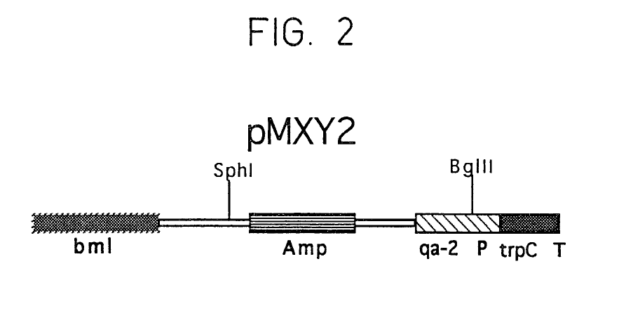

FIG. 2: Linear map of the pMXY2 plasmid. Plasmid genes are shown as box. bmI: beta-tubulin allele which is responsible for benilate resistance; Amp: ampicillin resistance; qa-2 P: qa-2 gene promoter; TrpC T: trpC gene terminator. SphI and BglII are restriction sites used for the plasmid recovery from the 627 mutant chromosomal DNA.

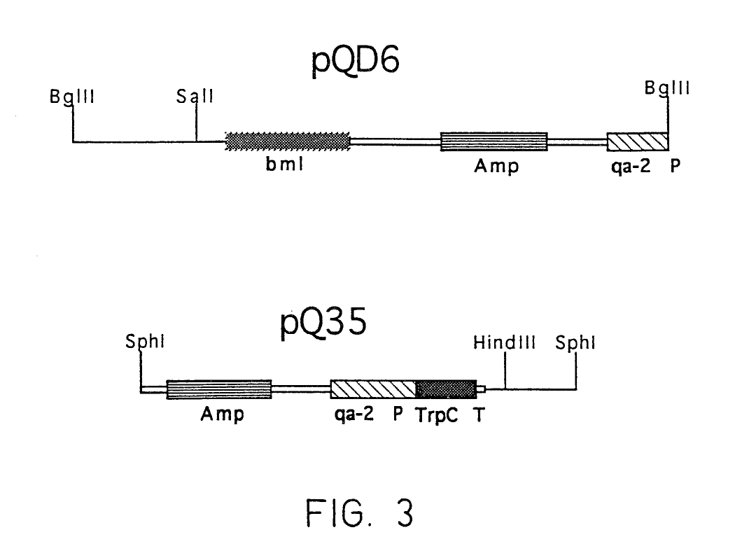

FIG. 3: Schematic representation of pQD6 and pQ35 plasmids. Restriction sites (BglII for pQD6 and SphI for pQ35) used for the recovery of the chromosomal DNA of the 627 strain are reported. Chromosomal sequences, flanking the integration site, are represented as segments. Restriction sites used to isolate DNA fragments used for probing the gene library are also represented.

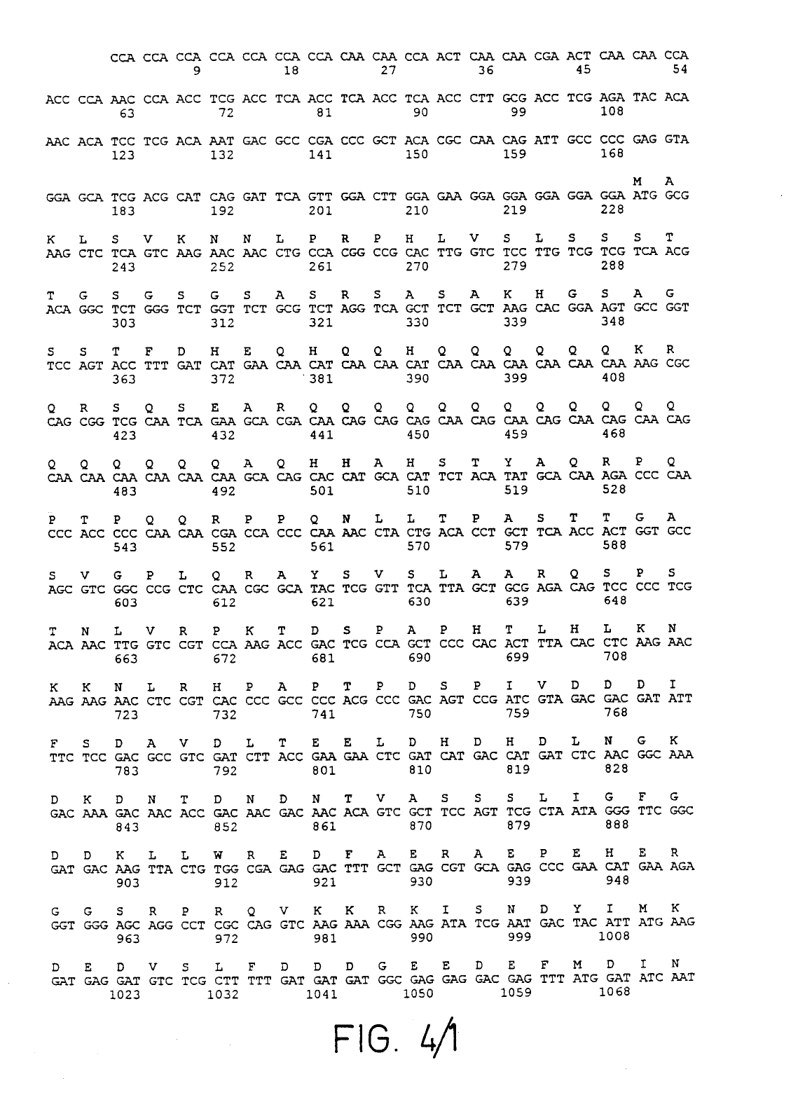

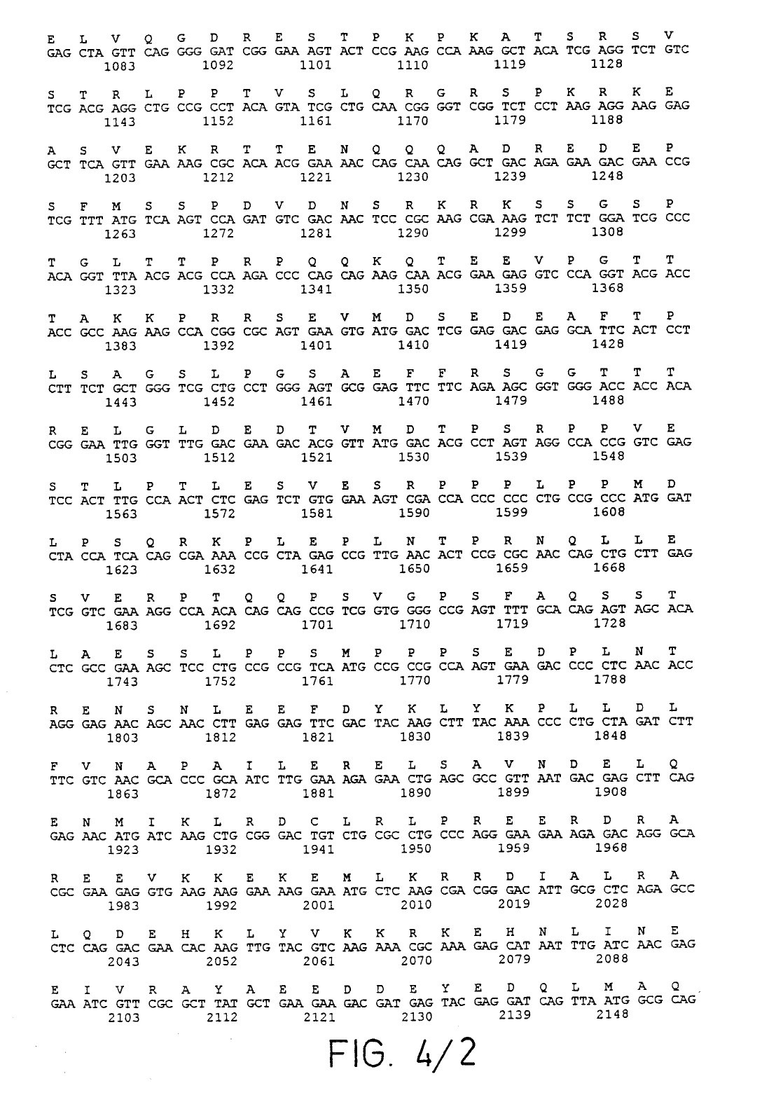

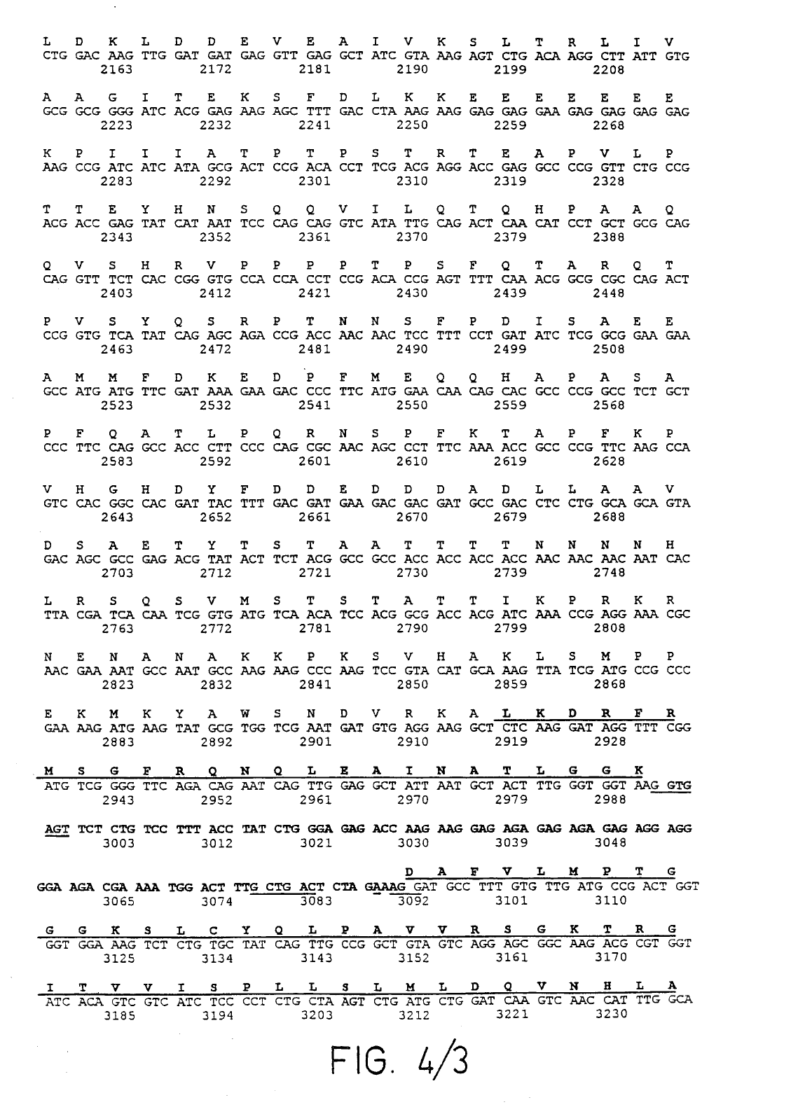

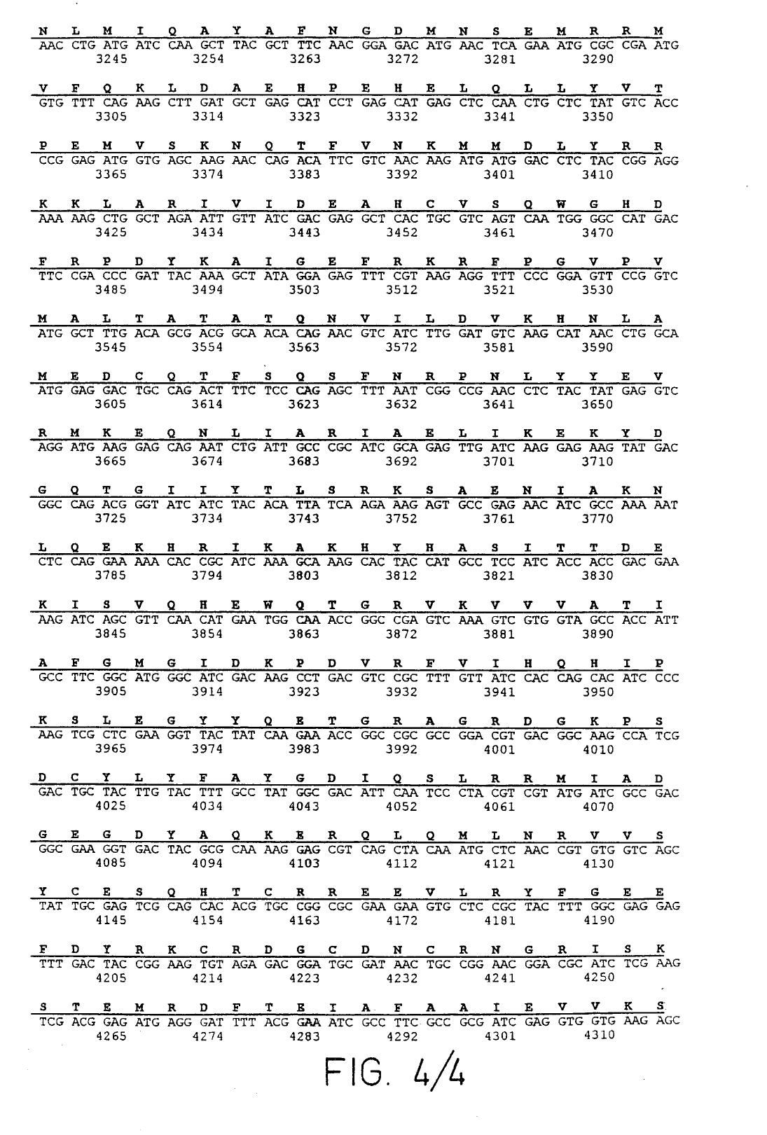

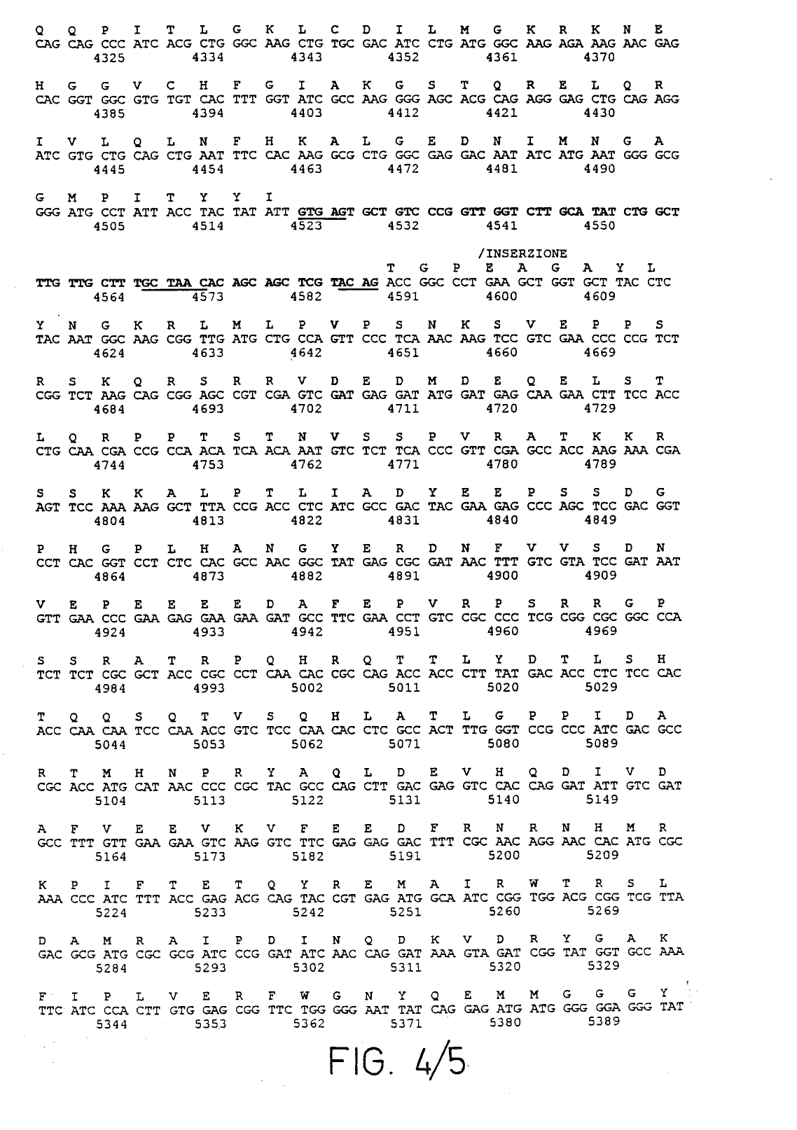

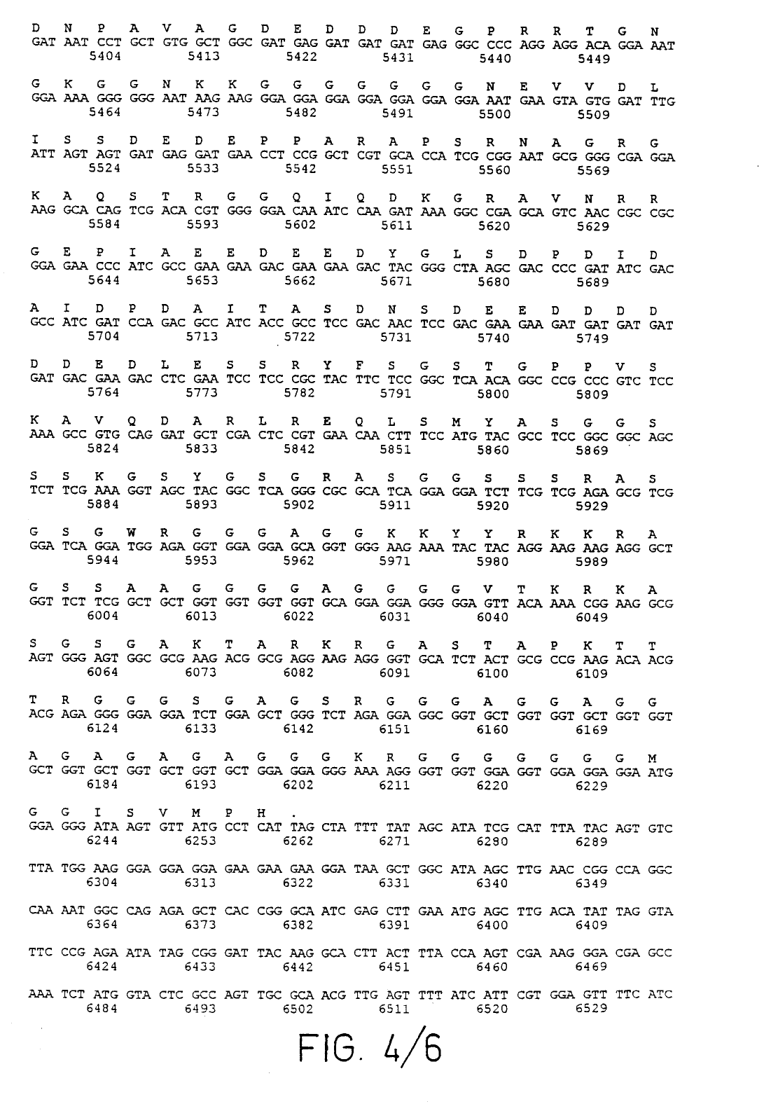

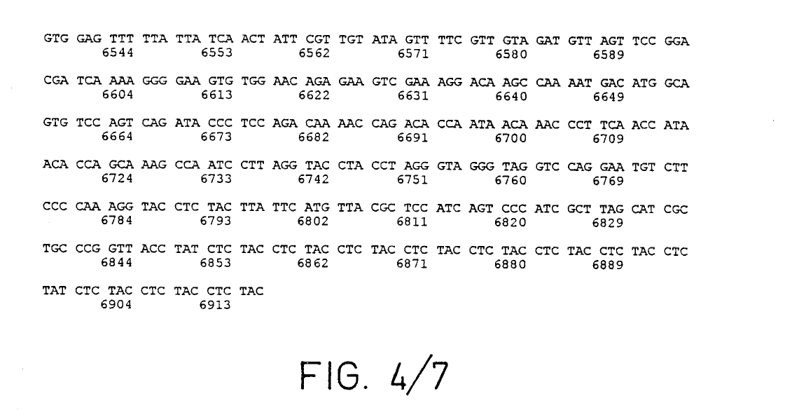

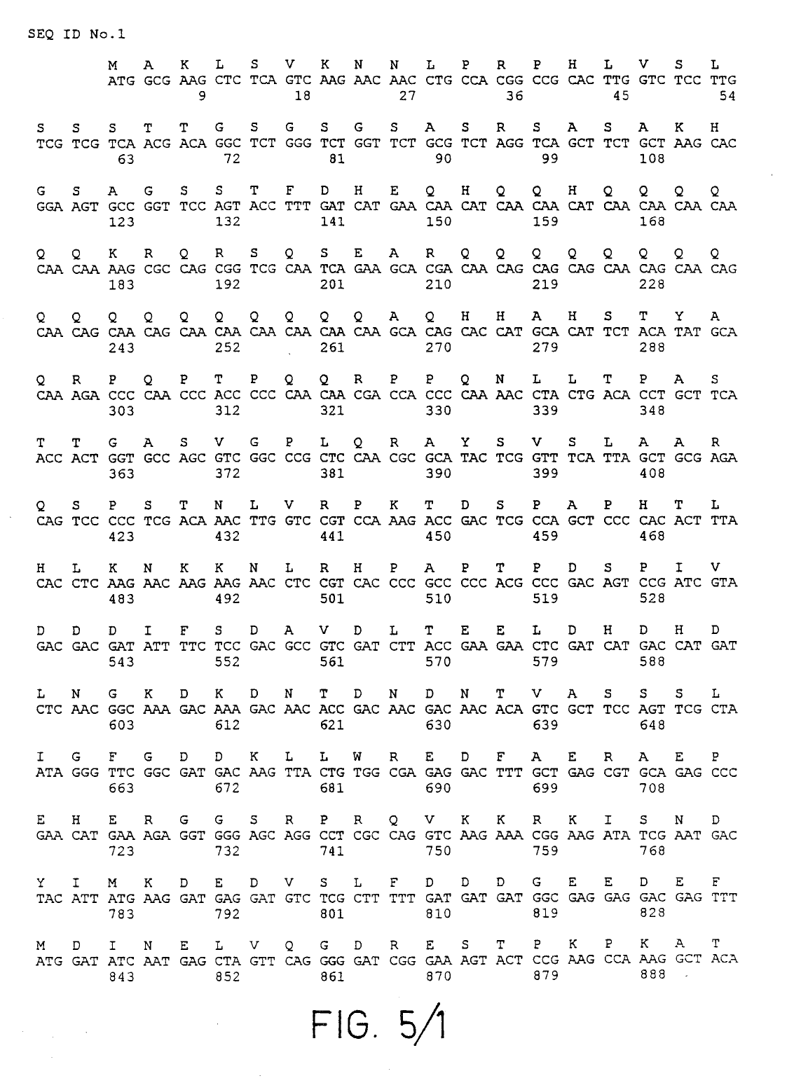

FIG. 4: Nucleotide sequence of the 6.9-Kb fragment containing the qde-3 gene and flanking sequences. The amino acid sequence is shown above the nucleotide sequence. The bold sequences represent two introns of 98 and 68 nt. In these regions the underlined nucleotides identify consensus sequences of the donor site, the acceptor site and the internal sequence or lariat. It is also represented the pMXY2 plasmid insertion site, in the 627 mutant, used for insertional mutagenesis. The portion encoding for the helicase domain is underlined.

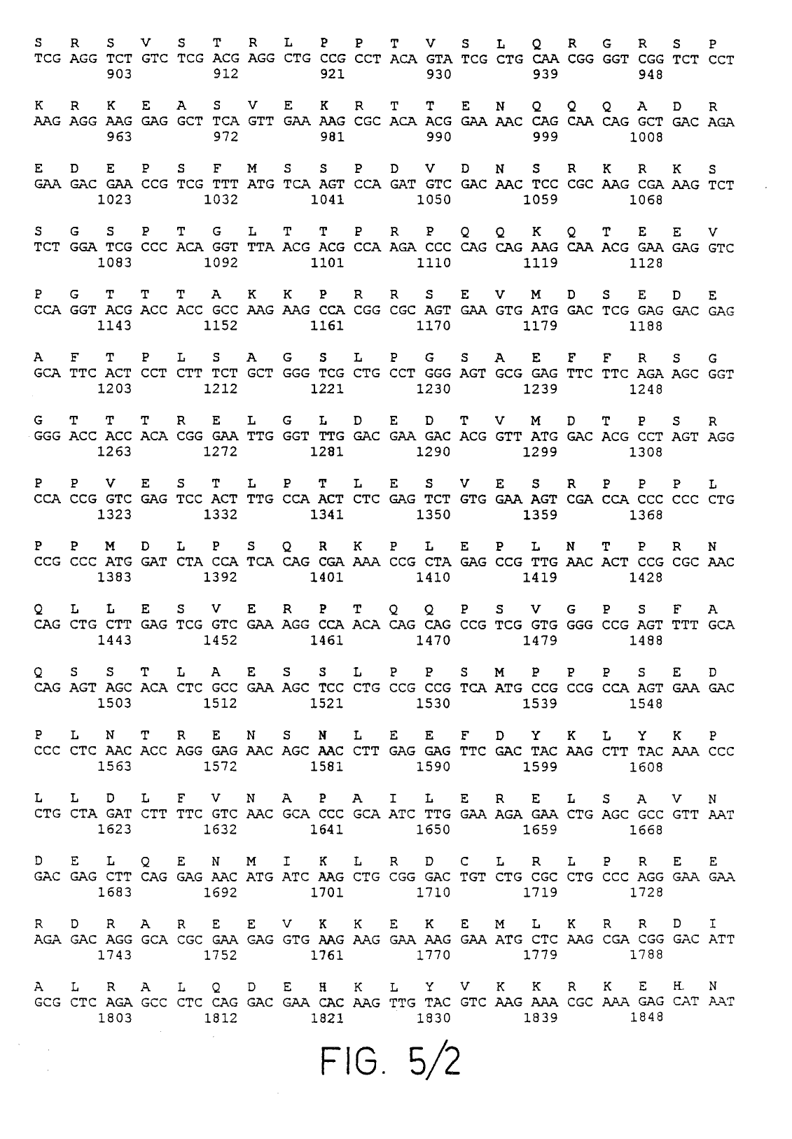

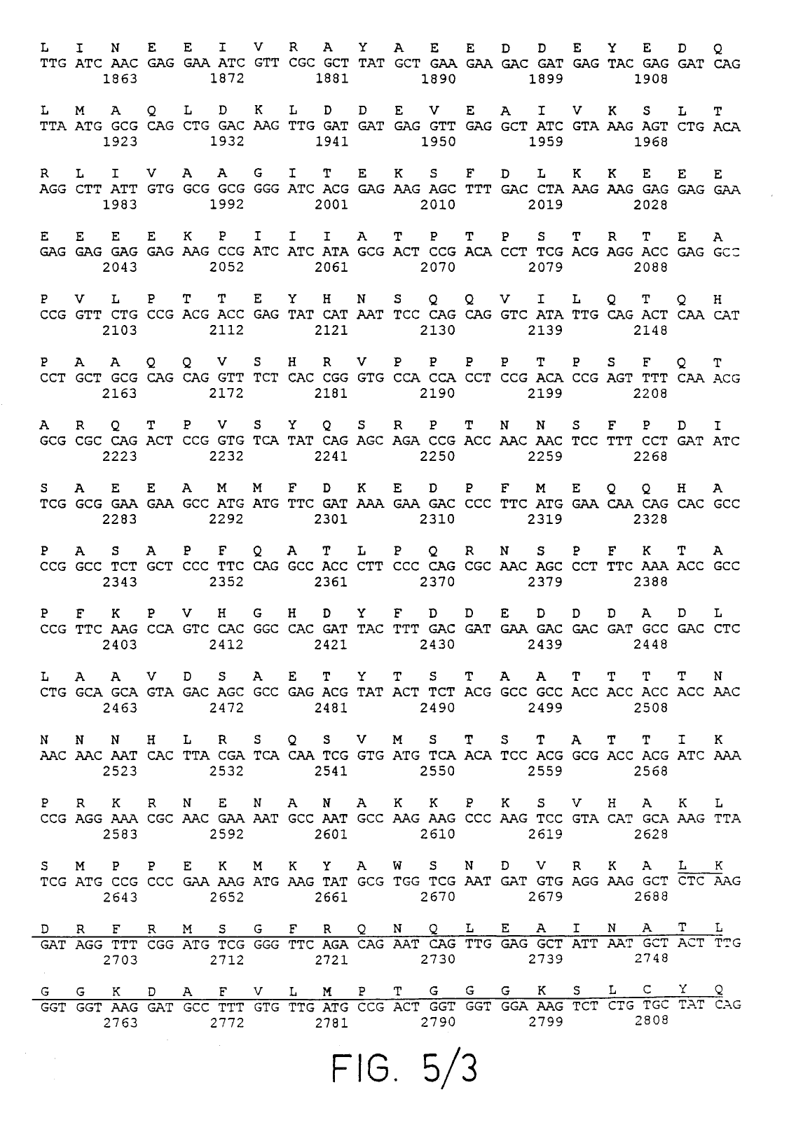

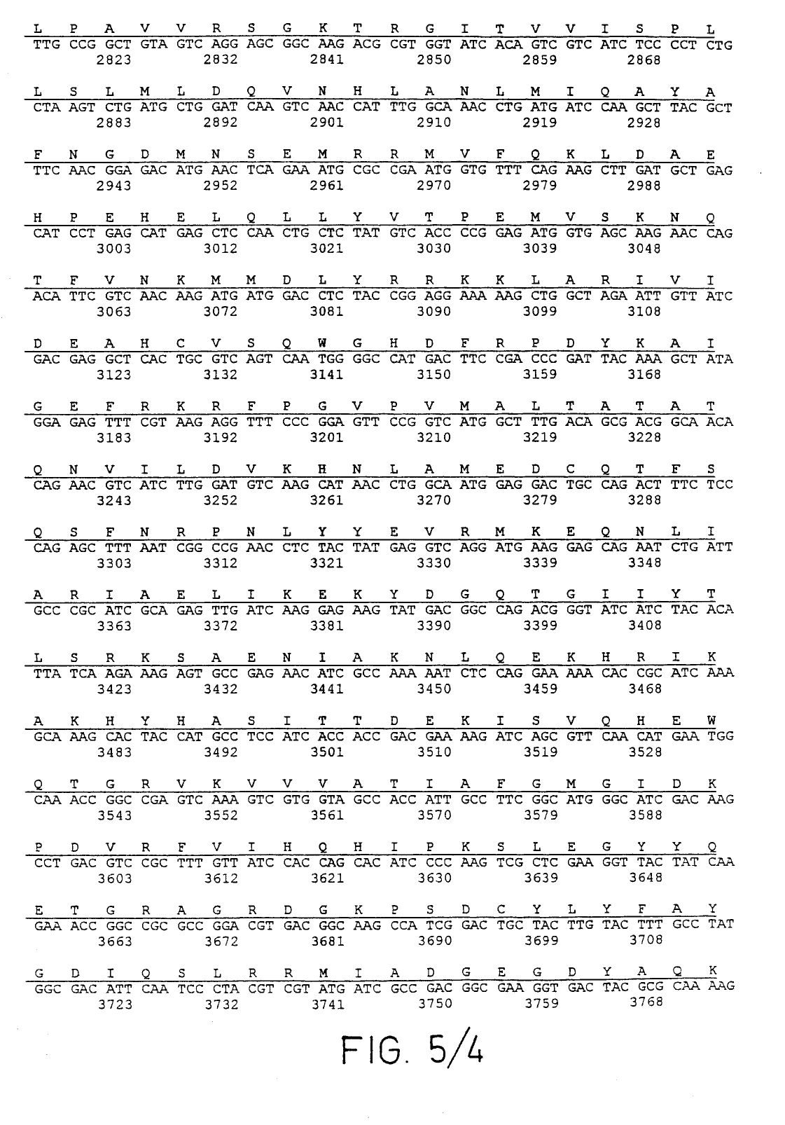

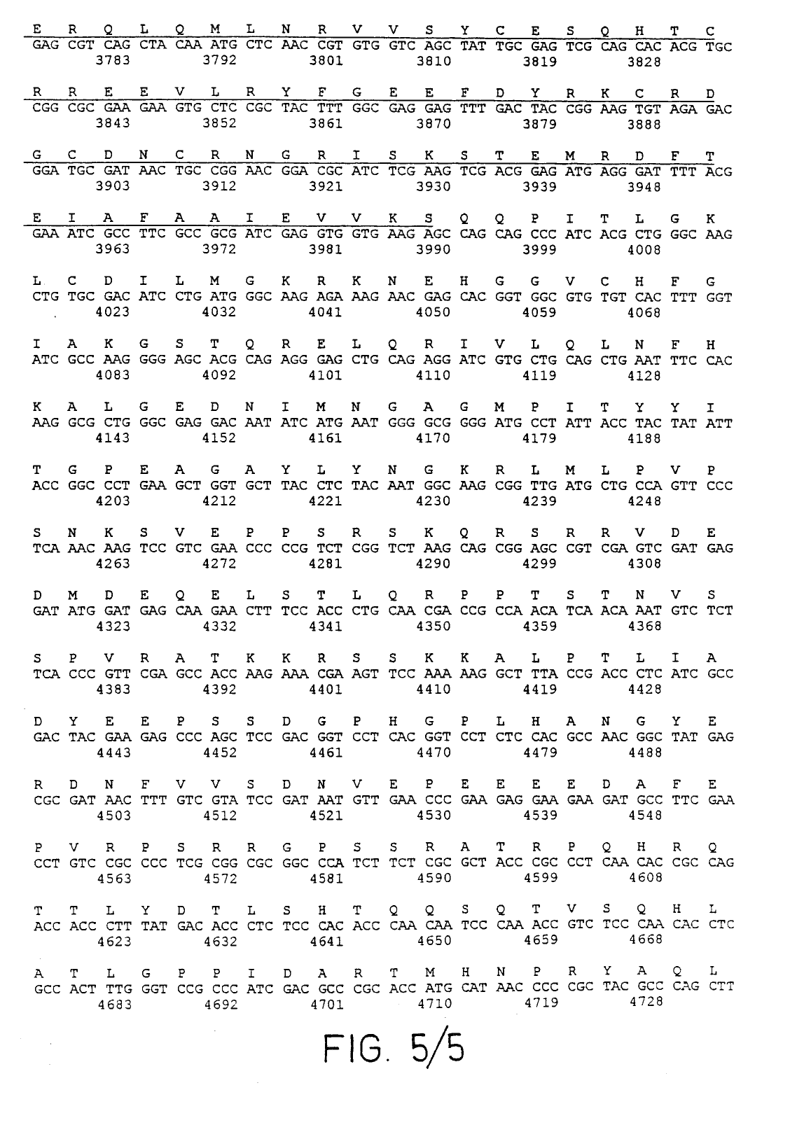

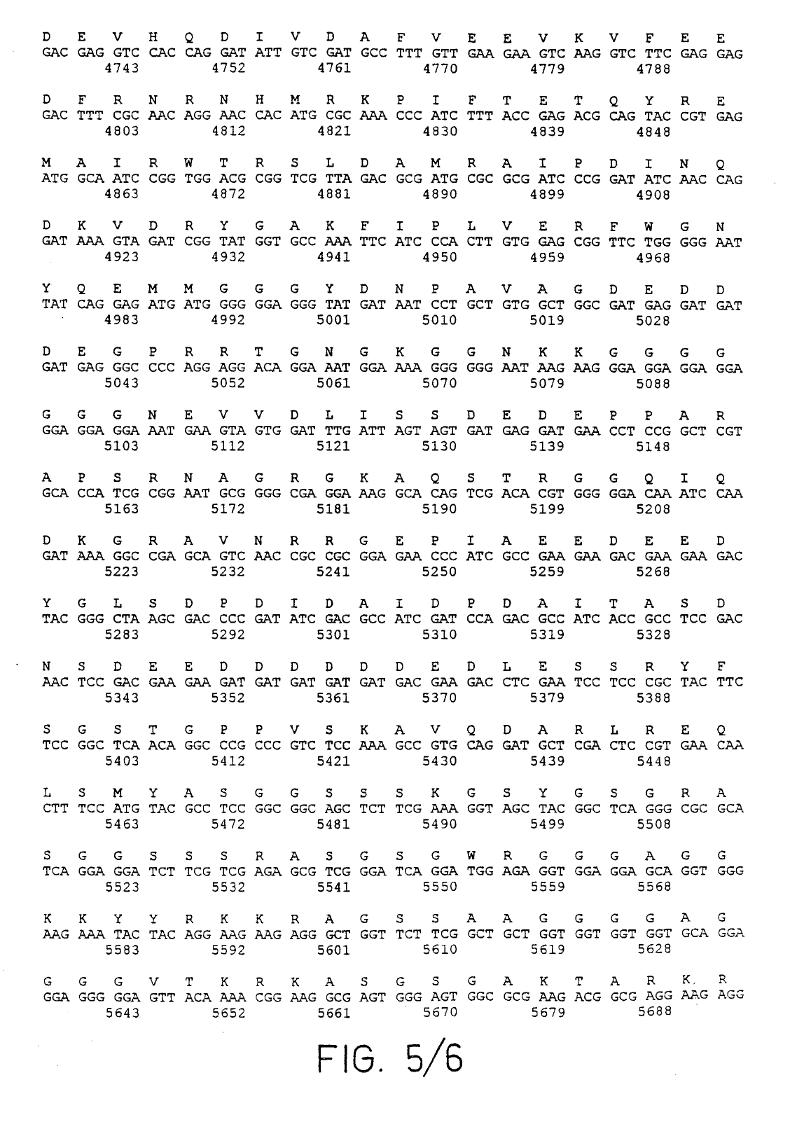

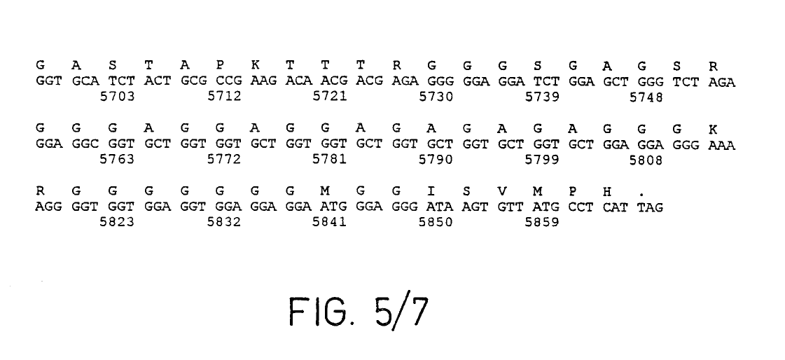

FIG. 5: Nucleotide sequence (SEQ ID No. 1) of the encoding portion reported in FIG. 4 and deduced amino acid sequence. Amino acids from 897 to 1330, which define the recQ DNA-helicase domain, are underlined.

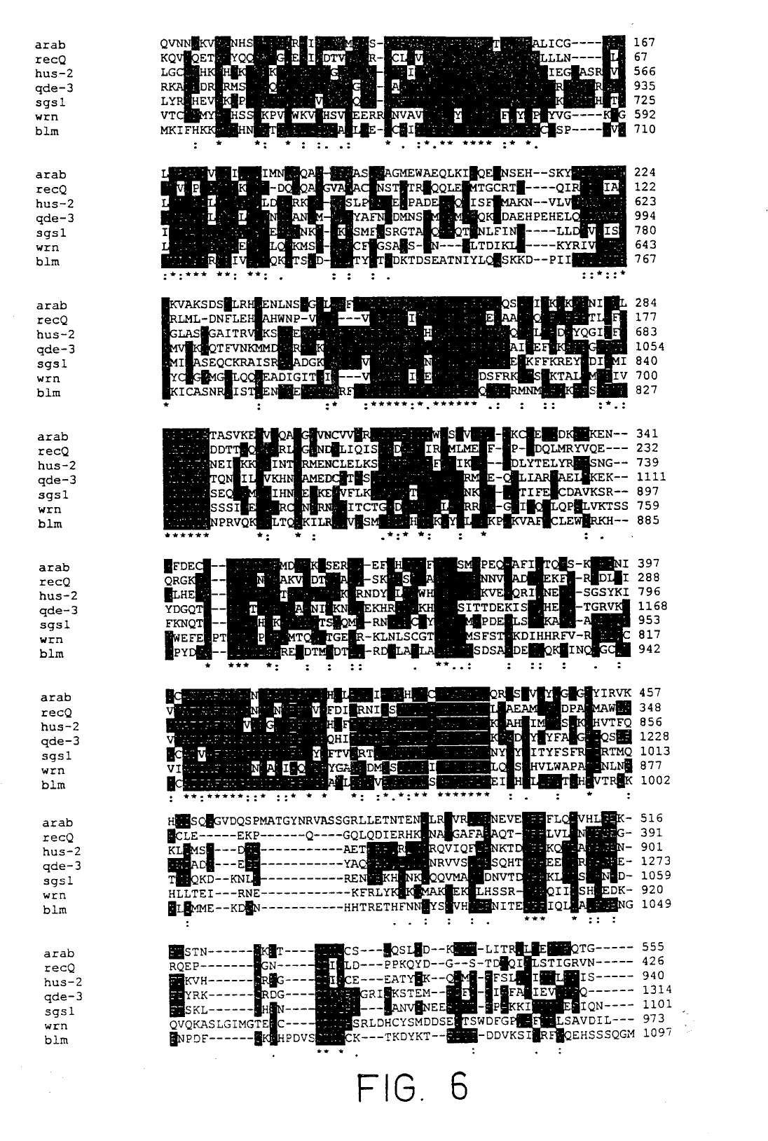

FIG. 6: Multiple alignment, at the conserved domains, among qde-3 and other proteins belonging to recQ family. arab recQ: A. thaliana isologous; E. coli recQ; S. pombe hus-2; S. cerevisiae sgs-1; human wrn: Werner syndrome; human blm: Bloom syndrome. Identical amino acids are shown in bold.

Materials and Methods

E. coli Strains

E. coli strain HB101 (F^{—}, hsdS20(rb^{—}, mb^{—}), supE44, recA13, ara14, proA2, rspL20(str^{r}), xyl-5) was used for cloning.

Neurospora crassa Strains and Growing Conditions

Neurospora crassa following strains, supplied by Fungal Genetic Stock Center (FGSC, Dpt. Of Microbiology, University of Kansas Medical Ctr. Kansas City, KA) were used:

Wild type (FGSC 987);

qa-2/aro9 (FGSC 3957A), (FGSC 3958a).

The 6XW strain (Cogoni et al., 1996) was obtained upon transformation of the FGCS 3958a strain with pX16 (Cogoni et al., 1996). This plasmid contains the qa-2 gene used as selective marker and the al-1 coding sequence.

The mutated strains M7, M20 (qde-1); M10, M11 (qde-2); M17, M18 (qde-3) are described in Cogoni and Macino, 1997.

The qde mutants were obtained by UV mutagenesis. As recipient the transforming strain (6xw) silenced at the albino-1 gene was used. qde mutants were selected for their ability to recover a wild type unsilenced phenotype and then classified in three different complementation groups. By analyzing the al-2 gene quelling frequency all of qde used mutants are defective for the general silencing mechanism.

Complementation assays with not forced heterocaryons were carried out according to Davis and DeSerres, 1970.

Plasmids and Libraries

The plasmid pMXY2, disclosed in Campbell et al., used for insertional mutagenesis was obtained from FGSC. The plasmid contains the Bm1 gene (allele responsible of the benilate drug resistance), that was used as selective marker after transformation. The genomic DNA containing the qde-2 gene was isolated from a N. Crassa gene library in cosmids. (Cabibbo et al., 1991).

N. crassa Transformation

Spheroplasts were prepared according to the Akins and Lambowitz (1985) protocol.

Southern Blot Analysis

Chromosomal DNA was prepared as disclosed by Irelan et al., 1993. 5 μg of genomic DNA were digested and blotted as reported in Maniatis et al.

DNA probes were: a) as to the al-1 gene the probe is represented by a XbaI-ClaI restriction fragment of pX16 (Cogoni et al., 1996); b) as to the BmI gene the probe is represented by the 2.6 Kb SalI fragment of pMXY2.

Northern Blot Analysis

N. crassa total RNA was extracted according to the protocol described by Cogoni et al., 1996. The mycelium was grown for two days at 30° C., then powdered in liquid nitrogen before RNA extraction. For Northern analysis 10 μg of RNA were formaldehyde denatured, electrophoresed on a 1% agarose, 7% formaldehyde gel, and blotted over Hybond N (Amersham) membranes. Hybridization was carried out in 50% formamide in the presence of 32 labeled DNA probe 1.5×10⁶ cpm/ml.

Results

Isolation of Silencing Mutant by Insertional Mutagenesis

Neurospora strain (6XW) wherein the albino-1 resident gene was steadily silenced was UV mutagenised, and qde (“quelling” deficient) mutants were isolated (Cogoni and Mancino 1997). The 6XW strain shows an albino phenotype due to the lack of carotenoid biosynthesis, as results by the silencing of the albino 1 gene expression (Schmidhauser et al., 1990). A mutation interfering with the silencing machinery is easily detectable by producing a wild type phenotype (bright orange) of the carotenoid biosynthesis. By means of complementation assays it was possible to establish that qde mutants belong to three complementation groups, indicating the presence of three genetic loci involved in the Neurospora silencing mechanism. In order to isolate the qde genes an insertional mutagenesis was carried out with the 6XW strain, previously used for UV mutagenesis. The insertional mutagenesis was carried out by transforming the 6XW strain with a plasmid, taking advantage of the fact that, after the transformation, plasmids are randomly inserted in the Neurospora crassa genome. The mutagenesis was carried out transforming the 6XW silenced strain with pMXY2 (see Materials and Methods) which contains the benilate resistance as selective marker. Transformed strains able to grow in the presence of benilate containing medium and showing a wild type phenotype for the carotenoid biosynthesis were selected. Out of 50.000 isolated independent transformed strains, a benilate resistant strain (627) was isolated, which showed the bright orange phenotype expected for a qde gene mutation. In order to verify that the silencing release was effectively due to a qde gene mutation and not to the loss of al-1, the genomic DNA of the strain 627 was extracted and digested with SmaI and HindIII restriction enzymes. After blotting, DNA was hybridized with a probe corresponding to the coding sequence of al-1. The SmaI site is present only once in the al-1 transgene containing plasmid and the digestion by using said enzyme produces a 5.5 Kb fragment corresponding to tandem arrayed al-1 transgenes, while a 3.1 Kb fragment is expected from the resident al-1 locus. FIG. 1 shows that the number of al-1 transgenic copies present in the 627 strain is comparable to that present in the silenced 6XW strain.

The 627 Strain Includes a Mutated qde3 Gene

The 627 strain was assayed in a heterokaryon assay with a wild type strain and with M7, M20 (qde-1) M10, M11 (qde-2) mutants (Cogoni and Macino, 1997). As shown in Table 1 the al-1 gene silencing is restored producing an albino phenotype in all of heterocaryons but M17 and M18. This behavior is consistent with the presence of a qde-3 gene recessive mutation in the 627 strain.

TABLE 1
Reciprocal heterokaryons among 627 mutant and previously
characterized qde mutants.

| | 627 | M7 | M20 | M10 | M11 | M17 | M18 |
|---|---|---|---|---|---|---|---|
| 627 | WT | AL | AL | AL | AL | WT | WT |
| M7 | | WT | WT | AL | AL | AL | AL |
| M20 | | | WT | AL | AL | AL | AL |
| M10 | | | | WT | WT | AL | AL |
| M11 | | | | | WT | AL | AL |
| M17 | | | | | | WT | WT |
| M18 | | | | | | | WT |

WT = heterokaryon with a wild type phenotype for carotenoid;
AL = heterokaryon with an albino phenotype wherein the al-1 gene silencing is restored.

Recovery of Sequences Flanking the PMXY2 Plasmid Integration Site

In order to recover sequences flanking the integration site or sites the following methodology was carried out. The 627 strain genomic DNA was restricted with SphI and BglII enzymes. As shown in the map of FIG. 2 the enzymes digest respectively upstream and downstream to the region containing both the ampicillin resistance gene and the origin of replication present in pMXY2. Subsequently the genomic DNA was ligated and the product used to transform E. coli cells. The screening was performed in an ampicillin-containing medium. pQD6 and pQ35 plasmids were recovered from BglII and SphI restricted chromosomal DNA, respectively (see FIG. 3). Two DNA fragments containing sequences flanking the integration site were isolated by using, respectively, BglII and SalI enzymes for pQD6, and SphI and HindIII enzymes for pQ35 (FIG. 3).

Isolation of Genomic Clones, their Subcloning and Complementation of the qde-3 Mutant

The two fragments from pQD6 and pQ35 plasmids were used to probe a Neurospora crassa genomic library in cosmids. Cosmids 6E8 and 54D7, both containing about 30 Kb genomic DNA inserts, were isolated. Both the probes recognize the same cosmids, thus indicating that the two flanking sequences are contiguous. Cosmids 6E8 and 54D7 were used in transformation experiments with M17 and M18 mutants. Both of cosmids are able to restore the al-1 gene silencing in the two mutants, determining an albino phenotype. Furthermore the introduction of same cosmids into the M10 (qde-2) or the M20 mutant (qde-1) is not effective to restore the silencing.

The 6E8 cosmid was used to subclone a 9 Kb SphI-SphI fragment. This subclone was used for transformation experiments and resulted to be able to complement the qde-3 phenotype, indicating that a qde-3 functional gene is present in this plasmid.

Isolation and Sequence of the qde-3 cDNA

The SphI-SphI region was sequenced, like the corresponding cDNA, by using RT-PCR. The latter sequence was used to deduce the qde-3 amino acid sequence and map the introns therein. The qde-3 gene encodes for a 1900 aa. putative protein (200 KDa). The genomic clone contains two introns of 98 nt. and 68 nt., respectively. Intron acceptor and donor sequences were identified and correspond to described consensus sequences (FIG. 4). Furthermore the pMXY2 plasmid insertion site within the gene in the 627 transforming strain is indicated. The insertion site was deduced by analysis of pQD6 and pQ35 plasmid sequences.

The cDNA sequence is shown in FIG. 5 (SEQ ID No. 1), wherein the helicase domain containing 434 amino acids from 897 aa to 1330 aa is underlined.

The qde-3 Gene is Belonging to recQ Helicase DNA Family

The 1900 aa sequence was used to search in database of amino acid sequences, by using the BLASTP algorithm. Significant homologies were identified with 6 genes belonging to the reQ family, belonging to the helicase group containing the DEAH consensus sequence. FIG. 6 shows the homologous region sequence alignment of helicase domains, as defined in FIG. 5, among qde-3 and genes belonging to recQ helicase family. qde-3 shows the highest homology with hus-2 (55% amino acid identity) and the lowest homology with Wrn (40% identity).

Plant Expression Vector

The qde-3 gene was inserted, in a sense orientation, into a vector containing a plant expression “cassette”, including the 35S promoter and the PI-II “terminator” sequences. The vector also includes the Streptomyces hygroscopicus bar gene, which confers the phosphinotricine herbicide resistance to transformed plants. In an analogous vector, qde-3 was inserted in an anti-sense orientation with respect to the 35S promoter.

The obtained vectors can be utilized to over-express the qde-3 gene in plants, or to repress the gene expression of resident genes, which are homologous to qde-3, respectively.

Fungus Expression Vector

The qde-3 gene was inserted in a vector containing a fungal specific expression “cassette”, comprising the A. nidulans trpC gene promoter and terminator, both in a sense and an anti-sense orientation. In addition the vector contains the bacterial hph gene, which confers the hygromicine drug resistance. The sense plasmid can be used to over express the qde-3 gene, whereas the anti-sense plasmid is used to repress the expression of qde-3 homologous genes in various fungine species.

Mammalian Expression Vector

The qde-3 gene was inserted in a vector containing a mammalian specific expression “cassette”, including the cytomegalovirus (CMV) promoter and SV40 termination and polyadenylation sequences both in a sense and anti-sense orientation. The vector includes also the neomicine phototransferase gene, as marker for mammalian cell selection. The sense plasmid can be used to over express the qde-3 gene, whereas the anti-sense plasmid can be used to repress the expression of qde-3 homologous genes in various mammalian species.

### BIBLIOGRAPHY

Akins, R. A. and Lambowitz A. M. (1985) Mol. Cell. Biol. 5:2272-2278

Baulcombe, D. C. (1996) Plant Mol. Biol. 32, 79-88.

Cogoni, C. et al. (1996) EMBO J. 15, 3153-3163

Cogoni, C. and Macino, G. (1997) Proc. Natl. Acad. Sci. U.S.A. 94: 10223-10238.

Cabibbo, A. et al. (1991) Fungal Genetic Newsl., 38: 68-70.

Davis, R. H. and De Serres, F. J. (1970) methods Enzymol. 17: 79-143.

de Carvalho Niebel, F. et al. (1995), Plant Cell 347-358.

Dehio, C., and Schell, J. (1994). Proc. Natl. Acad. Sci. U.S.A. 91: 5538-5542.

Dorer, D. R. and Henikoff, S. (1994). Cell, 993-1002.

Elkind, Y. Et al. (19990) Proc. Natl. Acad. Sci. U.S.A. 87: 9057-9061.

Ellis, N. A. et al. (1995) Cell 83:655-666.

Flavell, R. B. (1994) Proc. Natl. Acad. Sci. U.S.A. 91: 3490-3496.

Garrick D., et al. (1998) Nature Genetics 18, 56-59.

Gray, M. D. et al. (1997) Nature Genetics 17, 100-103.

Irelan, J. et al. (1993) Fungal Genetics Newsl. 40: 24.

Lindbo, J. A. et al. (1993) Plant Cell 5: 1749-1759.

Maniatis, S. T. et al. (1982) Molecular Cloning-A Laboratory Manual, Cold Spring Harbor, N.Y.: Cold Spring Harbor Laboratory Press.

Mittelstein Scheid, 0. Et al. (1994) Mol. Gen. Genet. 244: 325-330.

Orbach, M. J. et al. (1986) Mol. Cell. Biol., 6, 2452-2461.

Pal-Bhadra, M., et al., (1997). Cell 90, 479-490.

Palauqui, J. C. et al., (1997) EMBO J. 16: 4738-4745.

Pirrotta, V. (1997). TIG 13, 314-318.

Schmidhauser, T. J. et al., Mol. Cell. Biol. 10: 5064-5070

Stam, M. et al. (1997) Annals of Botany 79:3-12

Stewart, E. et al., (1997) EMBO J. 10:2682-2692

van Blokland, R. et al. (1994), Plant, 6, 861-887.

Vaucheret, H. (1993), C.R. Acad. Sci. Paris, Sciences de la vie/Life sciences 316, 1471-1483.

Wallrath, L. L. and Elgin, S. C. R. (1995). Genes & Development 9, 1263-1277.

Watt, P. M. et al. (1995). Cell, 81: 253-260.

Watt, P. M. et al., (1996). Genetics 144: 935-945.

Yu, C. E. et al. (1996). Science 272: 258-262.

1. An isolated nucleic acid molecule encoding for a protein characterized in having a silencing activity and in comprising a recQ helicase domain, wherein the domain is at least 30% homologous with the amino acid sequence from aa. 897 to aa. 1330 of SEQ ID No.1.

2. An isolated nucleic acid molecule encoding for a protein characterized in having a silencing activity and comprising a recQ helicase domain according to claim 1, wherein the domain is at least 40% homologous with the amino acid sequence from aa. 897 to aa. 1330 of SEQ ID No.1.

3. An isolated nucleic acid molecule encoding for a protein characterized in having a silencing activity and comprising a recQ helicase domain according to claim 2, wherein the domain is at least 60% homologous with the amino acid sequence from aa. 897 to aa. 1330 of SEQ ID No.1.

4. An isolated nucleic acid molecule encoding for a protein characterized in having a silencing activity and comprising a recQ helicase domain according to claim 3, wherein the recQ helicase domain has the amino acid sequence from aa. 897 to aa. 1330 of SEQ ID No.1.

5. An isolated nucleic acid molecule encoding for a protein characterized in having a silencing activity and comprising a recQ helicase domain according to claim 4, wherein said nucleotide sequence encodes for a protein having the amino acid sequence of SEQ ID No. 1, or functional portions thereof.

6. An isolated nucleic acid molecule encoding for a protein characterized in having a silencing activity and comprising a recQ helicase domain according to claim 5, wherein said nucleotide sequence is the sequence of SEQ ID No. 1 or its complementary sequence.

7. Expression vector comprising, under the control of a promoter that directs the expression of genes in bacteria, the isolated nucleic acid molecule according to any one of claims 1-6.

8. Expression vector comprising, under the control of a promoter that directs the expression of genes in plants or in specific plant organs, the isolated nucleic acid molecule according to any one of claims 1-6, both in a sense and anti-sense orientation.

9. Expression vector comprising, under the control of a promoter that directs the expression of genes in fungi, the isolated nucleic acid molecule according to any one of claims 1-6 both in a sense and anti-sense orientation.

10. Expression vector comprising, under the control of a promoter that is expressed in animals, the isolated nucleic acid molecule according to any one of claims 1-6 both in a sense and anti-sense orientation.

11. Prokaryotic organism transformed by using the expression vector active in bacteria according to claim 7.

12. Plants or a specific plant organ transformed by using the expression vector active in plants according to claim 8.

13. Plant mutated at the nucleotide sequence according to any one of claims 1-6 having a reduced or inhibited silencing activity.

14. Fungus transformed by using the expression vector active in fungi according to claim 9.

15. Non-human animal transformed by using the expression vector active in animals according to claim 10.

16. Non-human animal mutated at the nucleotide sequence according to any one of claims 1-6 having a reduced or inhibited silencing activity.

17. Protein characterized in having a silencing activity and comprising a recQ helicase domain wherein the domain is at least 30% homologous to the amino acid sequence from aa. 897 to aa. 1330 of SEQ ID No.1.

18. Protein characterized in having a silencing activity and comprising a recQ helicase domain according to claim 17 wherein the domain is at least 40% homologous to the amino acid sequence from aa. 897 to aa. 1330 of SEQ ID No.1.

19. Protein characterized in having a silencing activity and comprising a recQ helicase domain according to claim 18 wherein the domain is at least 60% homologous to the amino acid sequence from aa. 897 to aa. 1330 of SEQ ID No.1.

20. Protein characterized in having a silencing activity and comprising a recQ helicase domain according to claim 19 wherein the domain is the amino acid sequence from aa. 897 to aa. 1330 of SEQ ID No.1.

21. Protein characterized in having a silencing activity and comprising a recQ helicase domain according to claim 21 comprising the amino acid sequence of SEQ ID. No.1 or functional portions thereof.

22. Use of the nucleotide sequence according to any one of claims 1-6 to modulate the gene silencing in plants, animals and fungi.

23. Use of the nucleotide sequence according to any one of claims 1-6 to potentiate the antiviral-response in a plant.
